(19) United States
(12) Patent Application Publication
(10) Pub. No.: US 2026/0053957 A1
(43) Pub. Date: Feb. 26, 2026

(54) FREEZE-DRIED NANODROPLETS WITH FLUORINATED COMPOUND

(71) Applicant: Bracco Suisse SA, Cadempino (CH)

(72) Inventors: Romain MELICH, Choisy (FR); Charles BERGER, Montpellier (FR); David LAZARUS, Saint-Julien-en-Genevois (FR); Philippe BUSSAT, Pers-Jussy (FR); Samir CHERKAOUI, Feigeres (FR)

(73) Assignee: Bracco Suisse SA, Cadempino (CH)

(21) Appl. No.: 19/102,500

(22) PCT Filed: Aug. 11, 2023
(86) PCT No.: PCT/EP2023/072329

§ 371 Date: Feb. 10, 2025

(30) Foreign Application Priority Data
Aug. 12, 2022 (EP) 22190311.5
Jun. 19, 2023 (EP) 23180043.4

(51) Int. Cl. A61K 49/22; A61K 9/19

(52) U.S. Cl. CPC A61K 49/227; A61K 9/19

(57) ABSTRACT

The present invention generally relates to the field of ultrasound contrast-agents (USCA). In particular, it relates to a freeze-dried composition comprising an amphiphilic lipid compound comprising a phospholipid, a fluorinated compound in liquid form and a freeze-drying protecting component which may be reconstituted for preparing a suspension of nanodroplets useful in diagnostic or therapeutic applications. It further relates to the method for the preparation of such freeze-dried composition.

### TECHNICAL FIELD

The present invention generally relates to the field of ultrasound contrast-agents (USCA). In particular, it relates to a freeze-dried composition comprising an amphiphilic lipid compound comprising a phospholipid, a fluorinated compound in liquid form and a freeze-drying protecting component, which may be reconstituted for preparing a suspension of nanodroplets filled with said fluorinated compound and stabilized by said amphiphilic lipid compound, useful in diagnostic or therapeutic applications. It further relates to a method for the preparation of such freeze-dried composition.

### BACKGROUND OF THE INVENTION

Phase-change contrast agents (PCCAs) or acoustically activated nanodroplets are receiving increased popularity in both ultrasound diagnostic and therapeutic delivery. Except for the core, often consisting of liquid perfluorocarbons, nanodroplets display similar composition to commercially available gas-filled microbubbles. Owing to Acoustic Droplet Vaporization (ADV) process, encapsulated droplets are converted into gas bubbles upon exposure to ultrasound energy beyond a vaporization threshold. In fact, ultrasounds act as a remote trigger to promote the vaporization of the droplets in a controllable, non-invasive and localized manner. Thanks to their smaller size compared to conventional microbubbles, nanodroplets display prolonged in vivo circulation and deep penetration into the tissues via the extravascular space. Moreover, below the vaporization threshold, they are ultrasonically stable with low acoustic attenuation and can be acoustically vaporized at the location of interest.

Perfluorocarbon nanodroplets (“PFC-NDs”) present a real potential as an extravascular ultrasound contrast agent in numerous diagnostic and therapeutic applications including sonopermeabilization, thermal ablation, blood-brain barrier (BBB) disruption, multimodal imaging modalities and allow passive (due to the enhanced permeability and retention (EPR) effect in the tumor tissues) or active targeting (by incorporating targeted ligands) for localized delivery of therapeutic drugs or genes. Another potentially valuable characteristic of PFC-NDs is their possible application for novel imaging strategies such as UltraSound Super-Resolution Imaging (also referred as UltraSound Localization Microscopy) since these agents can be activated and deactivated on demand by applying intermittent acoustic pulses.

PFC-NDs are usually composed by an encapsulating shell and a core filled with liquid perfluorocarbon (PFC). Currently, commonly used shell materials include soft shell materials, such as lipid and fluorinated surfactants, or hard-shell materials, such as proteins and polymers (Zhang, 2022).

Notwithstanding lipids are successfully adopted in the fabrication of ultrasound-responsive contrast agents (e.g. microbubbles and droplets), due to their properties such as elasticity and acoustic behavior, it has been reported that lipid shell nanodroplets lack stability after storage probably due to vesicle aggregation.

Melich, 2020 reports the use of rapid and controlled microfluidic mixing for the manufacturing of various types of PFC-NDs with different stabilizing shells. In this study, it was demonstrated that polymer-coated nanodroplets (PLGA) showed higher stability than the lipid shelled (DPPC:DSPE-PEG2000).

There is thus a need to develop a procedure for the long-term storage of lipid shelled nanodroplets. Freeze-drying, also known as lyophilization, is a complex and challenging process, widely used in the pharmaceutical industry that has the advantage of preserving pharmaceutical products under a dry form over several months. In fact, freeze-dried products display greater storage stability, extended shelf life and can be easily shipped.

Up to now, according to Applicant's knowledge, such technique has not been applied yet for preparing a freeze-dried composition from a suspension of nanodroplets stabilized by a lipid shell with a core comprising a fluorinated compound in liquid form.

As observed by the Applicant, the main challenges in preparing a freeze-dried composition of nanodroplets filled with a fluorinated compound are related to the need of avoiding the removal of volatile fluorinated compound during the freeze-drying process and to the substantial preservation of the characteristics of the initial suspension of nanodroplets, such as mean size, size distribution, amount of fluorinated compound and nanodroplets concentration.

The Applicant has now found that such initial characteristics can be preserved to an acceptable extent after the freeze-drying process.

Moreover, Applicant has demonstrated that it is possible to obtain a reconstituted suspension of lipid-stabilized nanodroplets characterized by a substantially unvaried amount of fluorinated compound, in respect of the amount prior to the lyophilization, by simply adding to the freeze-dried composition an aqueous solution without any further gas instillation.

### SUMMARY OF THE INVENTION

An aspect of the invention relates to a freeze-dried composition comprising
i) an amphiphilic lipid compound comprising a phospholipid,
ii) a fluorinated compound in liquid form and
iii) a freeze-drying protecting component
   which, upon reconstitution with a pharmaceutically acceptable liquid carrier, provides a suspension of nanodroplets comprising an inner core and an outer layer, wherein said inner core comprises said fluorinated compound in liquid form and said outer layer comprises said amphiphilic lipid compound.

In a preferred embodiment, the weight ratio between said freeze-drying protecting component and said fluorinated compound is higher than 10, preferably higher than 20, preferably higher than 30, preferably higher than 50, preferably higher than 60, preferably higher than 100, up to 250.

In a preferred embodiment, said fluorinated compound has a boiling point comprised between −70° C. and 160° C., preferably comprised between −50° C. and 100° C., more preferably comprised between −40° C. and 60° C.

Still more preferably said fluorinated compound is a perfluorocarbon selected from perfluoropentane, perfluorohexane, perfluorobutane, perfluoropropane or a mixture thereof.

In another embodiment, said freeze-drying protecting component is a saccharide, preferably a disaccharide selected from trehalose, sucrose, maltose or a mixture thereof, preferred being trehalose.

According to a further aspect of the invention relates to a method for preparing a freeze-dried composition as defined above comprising the steps of:
a) preparing an initial suspension comprising
   i) a plurality of nanodroplets comprising an amphiphilic lipid compound comprising a phospholipid and a fluorinated compound in liquid form and
   ii) a freeze-drying protecting component; and
b) freeze-drying said initial suspension
wherein said freeze-dried composition upon reconstitution with a pharmaceutically acceptable liquid carrier, provides a suspension of nanodroplets as above defined, wherein the amount of said fluorinated compound is from 50% to 100% of the amount of fluorinated compound comprised in the initial suspension of step a).

In a preferred embodiment, said reconstitution does not comprise an additional instillation of fluorinated compound.

In a further aspect, the invention relates to a method for preparing a reconstituted suspension of nanodroplets, said nanodroplets comprising an inner core and an outer layer, wherein said inner core comprises a fluorinated compound in liquid form and said outer layer comprises an amphiphilic lipid compound comprising a phospholipid, said method comprising the steps of:
a) preparing an initial suspension comprising
   i) a plurality of nanodroplets comprising an amphiphilic lipid compound comprising a phospholipid and a fluorinated compound in liquid form and
   ii) a freeze-drying protecting component;
b) freeze-drying said initial suspension to obtain a freeze-dried composition, and
c) reconstituting said freeze-dried composition with a pharmaceutically acceptable liquid carrier providing a suspension said nanodroplets wherein the amount of said fluorinated compound is from 50% to 100% of the amount of fluorinated compound comprised in the initial suspension of step a).
   In a preferred embodiment at step c) the reconstitution of said free-drying composition does not comprise an additional instillation of fluorinated compound.

Another aspect relates to a freeze-dried composition as defined above for use in a diagnostic and/or therapeutic treatment.

### FIGURES

FIG. 1 DLS size distribution of Composition 3T before (dashed line) and after (solid line) the freeze-drying procedure.

### DETAILED DESCRIPTION OF THE INVENTION

The present invention generally relates to a freeze-dried composition comprising an amphiphilic lipid compound comprising a phospholipid, a fluorinated compound in liquid form and a freeze-drying protecting component which, upon reconstitution with a pharmaceutically acceptable liquid carrier, provides a suspension of nanodroplets, the characteristics of which (i.e. sizes, size distribution, amount of fluorinated compound and NDs concentration) result to be substantially unvaried with respect to those of the suspension prior to freeze-drying.

Said nanodroplets are suitable as contrast agents in ultrasound imaging techniques, known as Contrast-Enhanced Ultrasound (CEUS) Imaging, or in ultrasound-mediated therapeutic applications, e.g. thermal ablation or for ultrasound mediated drug/gene delivery.

As observed by the Applicant, the main characteristics of said nanodroplets (e.g. sizes, size distribution, amount of fluorinated compound and NDs concentration) after a storage period in the form of suspension, may not be maintained and may progressively deteriorate. The shelf-life of such suspensions of nanodroplets comprising a fluorinated compound in a liquid form is thus relatively short for a pharmaceutical product and there is a need to develop a long-term storage procedure, able to preserve their initial characteristics, such as sizes, sizes distribution, amount of fluorinated compound and NDs concentration for longer times, e.g. months or years. The freeze-drying process is a suitable approach to obtain a dry form of nanodroplets filled with a fluorinated compound, with increased stability over time and with preserved initial characteristics. It has now been surprisingly found that said initial characteristics can be substantially preserved after the freeze-drying process.

### Definitions

The term “nanodroplet” indicates an assembly comprising an outer layer and an inner core, said outer layer comprising an amphiphilic lipid compound comprising a phospholipid and said inner core comprising a fluorinated compound in a liquid form. In said nanodroplets, the amphiphilic lipid compound is oriented in such a way that the hydrophobic portions of the amphiphilic lipid are located at a surface of the fluorinated compound of the inner core.

According to this invention, preparations of said nanodroplets have a z-average diameter comprised between 100 nm and 1000 nm, preferably between 120 and 800 nm, more preferably between 150 and 400 nm. In certain embodiments, said nanodroplets are “calibrated nanodroplets” indicating preparations of nanodroplets with a population of nanodroplets having a z-average as above defined and a polydispersity lower than 0.25, preferably lower than 0.20, more preferably lower than 0.15, even more preferably lower than 0.10.

Said “calibrated nanodroplets” are preferably obtained through microfluidic technique.

As used herein, the expression “freeze-dried composition” indicates any dry dosage form for long-term storage of said nanodroplets filled with a fluorinated compound in liquid form obtained through a freeze-drying process. Said freeze-dried composition can comprise one or more active ingredients and a freeze-drying protecting component. The expression “active ingredient” as used herein comprises the nanodroplets stabilizing materials, i.e. the amphiphilic lipid compound, and the core materials, i.e. the fluorinated compounds.

The freeze-dried composition typically comprises a fluorinated compound entrapped therein in a liquid form, in particular in the core of the freeze-died nanodroplets.

As used herein, the expression “freshly-prepared nanodroplets” refers to (a suspension of) nanodroplets filled with a fluorinated compound in a liquid form at the end of its preparation, for instance within few minutes (e.g. 5 minutes) from the last step of the preparation method.

As used herein, the expression “initial suspension of nanodroplets” refers to an aqueous suspension of nanodroplets further comprising a freeze-drying protecting component. Typically, after obtaining a suspension of freshly-prepared nanodroplets by any suitable preparation method (as detailed below), said initial suspension of nanodroplets may be obtained by admixing a freeze-drying protecting component in the form of a suitable aqueous solution to the suspension of nanodroplets. Said initial suspension is then submitted to a freeze-drying procedure for preparing a freeze-dried composition, as defined above. Said freeze-dried composition is then reconstituted to a liquid form before being administered in vivo.

As used herein, the expression “reconstituted suspension of nanodroplets” refers to the suspension of nanodroplets comprising an inner core and an outer layer, wherein said inner core comprises a fluorinated compound in liquid form and said outer layer comprises an amphiphilic lipid compound comprising a phospholipid, said suspension obtained by reconstituting said freeze-dried composition with a pharmaceutically suitable liquid carrier (as defined below).

According to the present invention, the initial characteristics, such as sizes, sizes distribution, amount of fluorinated compound and NDs concentration possessed by said initial suspension of nanodroplets can be substantially preserved after the freeze-drying process by using suitable freeze-drying protecting component. It follows that the characteristics of said reconstituted suspension of nanodroplets filled with a fluorinated compound are substantially unvaried in respect of those characterizing the initial suspension.

### Freeze-Drying Protecting Components

The initial characteristics of the nanodroplets filled with a fluorinated compound in liquid form and stabilized by amphiphilic lipid compounds are particularly preserved when using a freeze-drying protecting component.

As used herein, the expression “freeze-drying protecting component” refers to a component suitable for freeze-drying, which is included in the suspension of nanodroplets before the freeze-drying process thereof. The term “freeze-drying protecting component” designates any compound added to protect the active ingredient during any phase of the freeze-drying process. Examples of suitable freeze-drying protecting components are polyethylene glycols (PEG), polyols, saccharides, surfactants, buffers, amino acids, chelating complexes, inorganic salts and mixtures thereof. Preferably said freeze-drying protecting component comprises polyethylene glycols (PEG), polyols, saccharides or a mixture thereof.

According to an embodiment of the invention, said freeze-drying protecting component comprises a saccharide.

The term “saccharide” has its standard meaning in the field of chemistry. Saccharides, also called carbohydrates, are molecular compounds made from just three elements: carbon, hydrogen and oxygen. The simplest saccharides are called monosaccharides and they are the building units for bigger saccharides, such as disaccharides, trisaccharide and polysaccharides.

Preferably, said saccharide is a disaccharide.

Disaccharides (C₁₂H₂₂O₁₁) are sugars composed of two monosaccharide units that are joined by a glycosidic bond. This latter is a covalent bond formed from the reaction of the anomeric carbon of one cyclic monosaccharide with the OH group of a second monosaccharide. Disaccharides differ from one another in their monosaccharide constituents and in the specific type of glycosidic linkage connecting them. Examples of disaccharides include trehalose, sucrose, maltose and lactose.

In the present invention, preferably the freeze-drying protecting component is a disaccharide selected from trehalose, sucrose, maltose or a mixture thereof. Particularly preferred is trehalose.

### Amphiphilic Lipid Compound

In the present description and claims, the expression “amphiphilic lipid compound” has its conventional meaning in the chemical field and refers to an organic lipid compound, comprising a hydrophilic moiety and a hydrophobic moiety, suitable for forming the stabilizing layer (i.e. outer layer) of the nanodroplets. In said nanodroplet, the amphiphilic lipid compound molecules are oriented in such a way that the hydrophobic portions are located at the surface of the liquid fluorinated compound placed in the inner core.

The disclosed nanodroplets are generally stabilized by one or more amphiphilic lipid compound comprising a phospholipid. Other suitable amphiphilic lipid compounds comprise, for instance lysophospholipids; fatty acids, such as palmitic acid, stearic acid, arachidonic acid or oleic acid; lipids bearing polymers, such as chitin, hyaluronic acid, polyvinylpyrrolidone or polyethylene glycol (PEG), also referred as “pegylated lipids”; lipids bearing sulfonated mono- di-, oligo- or polysaccharides; cholesterol, cholesterol sulfate or cholesterol hemisuccinate; tocopherol hemisuccinate; lipids with ether or ester-linked fatty acids; diacetyl phosphate; dicetyl phosphate; ceramides; polyoxyethylene fatty acid esters (such as polyoxyethylene fatty acid stearates), polyoxyethylene fatty alcohols, polyoxyethylene fatty alcohol ethers, polyoxyethylated sorbitan fatty acid esters; esters of sugars with aliphatic acids including sucrose laurate, fructose laurate, sucrose palmitate, sucrose stearate, glycerol or glycerol monoesters with fatty acids, including glycerol monopalmitate, glycerol monostearate, glycerol monomyristate or glycerol monolaurate; long chain alcohols including n-decyl alcohol, lauryl alcohol, myristyl alcohol, cetyl alcohol, or n-octadecyl alcohol; 1,2-dioleyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine; alkylamines or alkylammonium salts, comprising at least one (C₁₀-C₂₀), preferably (C₁₄-C₁₈), alkyl chain, such as, for instance, N-stearylamine, N,N′-distearylamine, N-hexadecylamine, N,N′-dihexadecylamine, N-stearylammonium chloride, N,N′-distearylammonium chloride, N-hexadecylammonium chloride, N,N′-dihexadecylammonium chloride, dimethyldioctadecylammonium bromide (DDAB), hexadecyltrimethylammonium bromide (CTAB); tertiary or quaternary ammonium salts comprising one or preferably two (C₁₀-C₂₀), preferably (C₁₄-C₁₈), acyl chain linked to the N-atom through a (C₃-C₆) alkylene bridge, such as, for instance, 1,2-distearoyl-3-trimethylammonium-propane (DSTAP), 1,2-dipalmitoyl-3-trimethylammonium-propane (DPTAP), 1,2-oleoyl-3-trimethylammonium-propane (DOTAP), 1,2-distearoyl-3-dimethylammonium-propane (DSDAP), (N-(4-carboxybenzyl)-N,N-dimethyl-2,3-bis(oleoyloxy)propan-1-aminium (DOPAQ), (6Z,9Z,28Z,31Z)-Heptatriaconta-6,9,28,31-tetraen-19-yl 4-(dimethylamino)butanoate (DLin-MC3-DMA) or 2-[2,2-bis[(9Z,12Z)-octadeca-9,12-dienyl]-1,3-dioxolan-4-yl]-N,N-dimethylethanamine (DLin-KC2-DMA); and mixtures or combinations thereof.

The term “phospholipid” is intended to encompass any amphiphilic phospholipidic compound, the molecules of which can form a stabilizing film at the boundary interface of the aqueous phase with the fluorinated compound in the final nanodroplets suspension.

Examples of suitable phospholipids include esters of glycerol with one or preferably two (equal or different) residues of fatty acids and with phosphoric acid, wherein the phosphoric acid residue is in turn bound to a hydrophilic group, such as, for instance, choline (phosphatidylcholines—PC), serine (phosphatidylserines—PS), glycerol (phosphatidylglycerols—PG), ethanolamine (phosphatidylethanolamines—PE), inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing from 12 to 24 carbon atoms, preferably from 14 to 22; the aliphatic chain may contain one or more unsaturations or is preferably completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Preferably, saturated fatty acids such as myristic acid, palmitic acid, stearic acid and arachidic acid are employed.

Further examples of phospholipids are phosphatidic acids, i.e. the diesters of glycerol-phosphoric acid with fatty acids; sphingolipids such as sphingomyelins, i.e. those phosphatidylcholine analogues where the residue of glycerol diester with fatty acids is replaced by a ceramide chain; cardiolipins, i.e. the esters of 1,3-diphosphatidylglycerol with a fatty acid; glycolipids such as gangliosides GM1 (or GM2) or cerebrosides; glucolipids; sulfatides and glycosphingolipids.

As used herein, the term “phospholipids” include either naturally occurring, semisynthetic or synthetically prepared products that can be employed either singularly or as mixtures.

Examples of naturally occurring phospholipids are natural lecithins (phosphatidylcholine (PC) derivatives) such as, typically, soy bean or egg yolk lecithins.

Examples of semisynthetic phospholipids are the partially or fully hydrogenated derivatives of the naturally occurring lecithins. Preferred phospholipids are fatty acids di-esters of phosphatidylcholine, ethylphosphatidylcholine, phosphatidylglycerol, phosphatidic acid, phosphatidylethanolamine, phosphatidylserine, phosphatidylinositol or of sphingomyelin.

Examples of preferred phospholipids are, for instance, dilauroylphosphatidylcholine (DLPC), dimyristoylphosphatidylcholine (DMPC), dipalmitoylphosphatidylcholine (DPPC), distearoylphosphatidylcholine (DSPC), diarachidoylphosphatidylcholine (DAPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroyl-phosphatidylglycerol (DLPG) and its alkali metal salts, diarachidoylphosphatidyl-glycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid (DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoyl-phosphatidylethanolamine (DMPE), dipalmitoylphosphatidylethanolamine (DPPE), distearoylphosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoylphosphatidylserine (DMPS), diarachidoyl phosphatidylserine (DAPS), dipalmitoylphosphatidylserine (DPPS), distearoylphosphatidylserine (DSPS), dioleoylphosphatidylserine (DOPS), dipalmitoylsphingomyelin (DPSP), distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylph osphatidylinositol (DSPI), dioleoyl-phosphatidylinositol (DOPI).

Suitable phospholipids further include phospholipids modified by linking a hydrophilic polymer, such as polyethyleneglycol (PEG) or polypropyleneglycol (PPG), thereto. Preferred polymer-modified phospholipids include “pegylated phospholipids”, i.e. phospholipids bound to a PEG polymer. Examples of pegylated phospholipids are pegylated phosphatidylethanolamines (“PE-PEGs” in brief) i.e. phosphatidylethanolamines where the hydrophilic ethanolamine moiety is linked to a PEG molecule of variable molecular weight (e.g. from 300 to 20000 daltons, preferably from 500 to 5000 daltons), such as DPPE-PEG (or DSPE-PEG, DMPE-PEG, DAPE-PEG or DOPE-PEG). For example, DPPE-PEG2000 refers to DPPE having attached thereto a PEG polymer having a mean average molecular weight of about 2000.

Particularly preferred phospholipids are DMPC, DPPC, DSPC, DMPG, DPPG, DMPA, DPPA, DMPS, DPPS and Ethyl-DPPC. Most preferred are DPPC, DMPA, DPPG.

Mixtures of phospholipids can also be used, such as, for instance, mixtures of DPPE and/or DSPE (including pegylated derivatives), DPPC, with DMPA, DPPA, DMPG, DPPG, or Ethyl-DPPC.

For instance, a mixture of phospholipids may include phosphatidylcholine derivatives, phosphatidic acid derivatives and pegylated phosphatidylethanolamine, e.g. DPPC/DMPA/DPPE-PEG, DPPC/DPPA/DSPE-PEG, DPPC/DPPG/DPPE-PEG, DPPC/DPPG/DSPE-PEG.

According to the present invention, the phospholipid can conveniently be used in admixture with any of the above listed amphiphilic compounds. Thus, for instance, lipids such as cholesterol, ergosterol, phytosterol, sitosterol, lanosterol, tocopherol, propyl gallate or ascorbyl palmitate, fatty acids such as myristic acid, palmitic acid, stearic acid, arachidic acid and derivatives thereof or butylated hydroxytoluene and/or other non-phospholipid compounds can optionally be added to one or more of the foregoing phospholipids. For instance, mixtures of amphiphilic lipid compounds comprising phospholipids and fatty acids can advantageously be used, including DPPC/DPPE-PEG/palmitic acid, DSPC/DPPE-PEG/palmitic acid, DPPC/DSPE-PEG/palmitic acid, DPPC/DPPE-PEG/stearic acid, or DPPC/DSPE-PEG/stearic acid.

In an embodiment said amphiphilic lipid compound is a mixture of amphiphilic lipid compounds comprising a phospholipid. Still more preferably said mixture further comprises a lipid bearing polymer, being preferred a pegylated phospholipid, as defined above.

According to a further embodiment, said mixture further comprises a fatty acid, preferred being palmitic acid.

### Fluorinated Compounds

As described above, the disclosed nanodroplets comprise an outer layer and an inner core, said outer layer comprising an amphiphilic lipid compound comprising a phospholipid and said inner core comprising a fluorinated compound in liquid form that allows for activation of the vesicle upon application of ultrasound (US).

In the present description and claims the term “fluorinated compounds” refers to a group of fluorine-containing compounds derived from (optionally substituted) hydrocarbons where hydrogen atoms have been partially or completely replaced by fluorine atoms, which are liquid or gas at room temperature.

The expression “optionally substituted” refers to presence of functional groups, such as amines, ethers and halogen-containing groups.

Suitable examples of fluorinated compounds are hydrofluorocarbons, both saturated and unsaturated, perfluorocarbons, fluorinated ethers, fluorinated ketones or perfluorinated nitriles. Preferably the fluorinated compound is a perfluorocarbon (PFC), i.e. a fluorinated hydrocarbon where all the hydrogen atoms are substituted with fluorine atoms.

Said fluorinated compound can be selected from fluorinated compound having a high boiling point, i.e. above room temperature (RT; 25° C.) that are in a liquid form at Standard Ambient Temperature and Pressure (SATP), namely at 25° C. and 1 atm (101.325 kPa), or fluorinated compounds having a low boiling point, i.e. below room temperature, referring to fluorinated compounds that are a gas at SATP (i.e. highly volatile fluorocarbons).

In an embodiment, said fluorinated compound has a boiling point comprised between −70° C. and 160° C., preferably comprised between −50° C. and 100° C., more preferably comprised between −40° C. and 60° C.

In a preferred embodiment, said fluorinated compound is a perfluorocarbon.

Liquid fluorinated compounds are characterized by a boiling point higher than 250, e.g. comprised between 25° C. and 160° C. In the present invention, the fluorinated compounds are preferably characterized by a boiling point comprised between 25° C. and 100° C., still more preferably between 27° C. and 60° C.

Suitable examples of liquid fluorinated compounds are 1-Fluorobutane, 2-Fluorobutane, 2,2-Difluorobutane, 2,2,3,3-Tetrafluorobutane, 1,1,1,3,3-Pentafluorobutane, 1,1,1,4,4,4-Hexafluorobutane, 1,1,1,2,4,4,4-Heptafluorobutane, 1,1,2,2,3,3,4,4-Octafluorobutane, 1,1,1,2,2-Pentafluoropentane, 1,1,1,2,2,3,3,4-Octafluoropentane, 1,1,1,2,2,3,4,5,5,5-Decafluoropentane, 1,1,2,2,3,3,4,4,5,5,6,6-Dodecafluorohexane, or a mixture thereof.

Suitable examples of liquid perfluorocarbons are perfluoropentane, perfluorohexane, perlfluoroheptane, perfluorooctane, perfluorononane, perfluorodecalin, perfluorooctylbromide (PFOB), perfluoro-15-crown-5-ether (PFCE), perfluorodichlorooctane (PFDCO), perfluorononane (PFN), and 1,1,1-tris(perfluorotert-butoxymethyl)ethane (TPFBME), or a mixture thereof.

In an embodiment said perfluorocarbon is preferably perfluoropentane (PFP) (boiling point 29° C.) or perfluorohexane (PFH) (boiling point 57° C.).

Gaseous fluorinated compounds are characterized by a boiling point lower than 25° C., e.g. comprised between −70° C. and 25° C., at atmospheric pressure. In the present invention, the fluorinated compounds are preferably characterized by a boiling point comprised between −50° C. and 22° C., more preferably between −45° C. and 18° C., more preferably between −40° C. and 5°, still more preferably −5° C. and 5° C.

Suitable examples of gaseous fluorinated compounds are 1,1,1,2,3,3,3 heptafluoropropane, 1,1,1,2,2,3-Hexafluoropropane, 1,1,1,2,3,3-Hexafluoropropane, 1,1,1,3,3,3-Hexafluoropropane, 1,1,1,2,2,3,3,4,4 Nonafluorobutane, 1,1,1,3,3,3-Hexafluoro-2-(trifluoromethyl)propane, 1,1,1,2,2,3,3,4 Octafluorobutane or a mixture thereof.

Suitable examples of gaseous perfluorocarbons are perfluorocyclopropane, perfluoropropane, perfluorocyclobutane, perfluorobutane, perfluoroisobutane or a mixture thereof.

In an embodiment said gaseous perfluorocarbon is preferably perfluorobutane (boiling point −2° C.) or perfluoropropane (boiling point −37° C.).

In a further embodiment, said perfluorocarbon comprises a mixture of a liquid perfluorocarbon and a gaseous perfluorocarbon.

In a preferred embodiment, said perfluorocarbon is selected from perfluorohexane, perfluoropentane, perfluorobutane, perfluoropropane or a mixture thereof.

### Preparation of the Freeze-Dried Composition

A further aspect of the invention relates to a method for preparing a freeze-dried composition as above defined comprising the steps of:
a) preparing an initial suspension comprising
   i) a plurality of nanodroplets comprising an amphiphilic lipid compound comprising a phospholipid and a fluorinated compound in liquid form and
   ii) a freeze-drying protecting component; and
b) freeze-drying said initial suspension
wherein said freeze-dried composition upon reconstitution with a pharmaceutically acceptable liquid carrier, provides a suspension of nanodroplets, wherein the amount of said fluorinated compound is from 50% to 100% of the amount of fluorinated compound comprised in the initial suspension of step a).
   Step a) Preparing an Initial Suspension of Nanodroplets Filled with a Fluorinated Compound

According to the present invention, at step a) said initial suspension is prepared by using any preparation technique suitable to manufacture nanodroplets having a Z-average diameter comprised between 100 nm and 1000 nm, preferably between 120 and 800 nm, more preferably between 150 and 400 nm.

Example of preparation techniques include sonication, homogenisation, extrusion, microfluidic and microbubble condensation.

In a preferred embodiment, at step a) said suspension is preferably prepared by using a preparation technique suitable to manufacture calibrated nanodroplets having a polydispersity index (PDI) lower than 0.25, preferably lower than 0.20, more preferably lower than 0.15, even more preferably lower than 0.10, and a Z-average diameter comprised between 100 nm and 1000 nm, preferably between 120 and 800 nm, more preferably between 150 and 400 nm.

For instance, said preparation technique is selected from sonication, microbubble condensation or microfluidic.

In the present description and claims the expression “sonication” refers to a technology of manufacturing nanodroplets based on the emulsification of their components (i.e. lipid-shell material and fluorinated compound) by using ultrasounds in a continuous aqueous phase. For instance, this technique can be performed by using a sonication bath or a probe sonicator.

In an embodiment, at step a) said initial suspension is prepared by using sonication techniques.

Typically, the sonication technique comprises the following steps:
aⁱ) Preparing an aqueous mixture comprising an amphiphilic lipid compound comprising a phospholipid and a fluorinated compound;
bⁱⁱ) Submitting said mixture to ultrasound and
cⁱⁱⁱ) Obtaining an aqueous suspension of nanodroplets comprising an outer shell comprising an amphiphilic lipid compound and an inner core comprising a fluorinated compound in a liquid form.

At the end of the sonication method, the suspensions of nanodroplets can be directly admixed to a solution of freeze-drying protecting component, as described above, in order to obtain an initial suspension of nanodroplets as defined in the present invention.

In the present description and claims the expression “microbubbles condensation” refers to a technology of manufacturing nanodroplets based on generating microbubbles filled with a gaseous perfluorocarbon and then condensing this gaseous precursor into liquid core nanodroplets by cooling and/or applying pressure.

In another embodiment, at step a) said initial suspension is prepared by using microbubble condensation methods.

Typically, the microbubble condensation method comprises the following steps:
a*) preparing a suspension of gas-filled microvesicles, having an outer shell comprising an amphiphilic lipid compound comprising a phospholipid and an inner core comprising a fluorinated compound being in a gaseous form at room temperature and atmospheric pressure;
b*) condensing said gaseous fluorinated compound to a liquid fluorinated compound, and
c*) obtaining a suspension of nanodroplets comprising an outer shell comprising an amphiphilic lipid compound comprising a phospholipid and an inner core comprising a fluorinated compound in liquid form.

After the condensation, the inner core of the obtained nanodroplets is composed by a fluorinated compound in a liquid form independently from the temperature at which they are subsequently stored.

In other words, even if the storage is effected at a temperature above the boiling point of said fluorinated compound used for the manufacturing of the NDs, said fluorinated compound remains in a liquid state due to its incorporation into the outer stabilizing shell.

In one embodiment said fluorinated compound includes a gaseous fluorinated compound as above defined, having a boiling point that is below room temperature (25° C.).

Said suspension of gas-filled microvesicles can be advantageously prepared by using any method known in the literature, e.g. by reconstituting a freeze-dried product with a suitable pharmaceutically accepted solution to obtain a suspension of gas-filled microvesicles.

In an embodiment, step b*) of the disclosed microfluidic condensation method comprises cooling the suspension of gas-filled microvesicles under pressure until the encapsulated gas condenses into a liquid phase.

Suitable examples of temperatures and pressures to be applied in step b*) can be found in US20220000790.

In the present description and claims the expression “microfluidic technique” refers to a technology of manufacturing nanodroplets through a microfluidic cartridge designed to manipulate fluids in channels at the microscale.

Said microfluidic technique is a bottom-up approach, that is to say that the nanodroplets are obtained by assembling molecules (e.g. amphiphilic lipid compounds and fluorinated compounds) into larger nanostructures (i.e. calibrated nanodroplets).

In a preferred embodiment, at step a) said initial suspension is prepared by using microfluidic techniques.

More preferably said microfluidic technique is carried out by using a mixing device such as a microfluidic cartridge equipped by a staggered herringbone micromixer or toroidal mixer.

A description of a microfluidic cartridge suitable for the microfluidic process of the present invention is described in WO2022101365A1 (Bracco Suisse SA).

In a still more preferred embodiment, the method for preparation of step a) comprises the following steps:
A) Preparing an aqueous phase;
B) Preparing an organic phase;
wherein:
   said aqueous phase comprises an amphiphilic lipid compound comprising a phospholipid and the organic phase comprises a fluorinated compound; or
   said organic phase comprises an amphiphilic lipid compound comprising a phospholipid and a fluorinated compound;
C) Injecting said aqueous phase into a first inlet and said organic phase into a second inlet of a microfluidic cartridge thereby mixing said aqueous phase and said organic phase in a mixing device of the microfluidic cartridge, wherein the operating pressure into said microfluidic cartridge is lower than 7000 kPa, to obtain an aqueous suspension of calibrated nanodroplets;
D) Collecting the aqueous suspension of calibrated nanodroplets from the exit channel of the microfluidic cartridge, and
E) Diluting the collected aqueous suspension of calibrated nanodroplets.

In an embodiment, said method further comprises the step F) of washing the obtained aqueous suspension of calibrated nanodroplets.

The expression “calibrated nanodroplets” relates to preparations of nanodroplets with a population of nanodroplets having a z-average diameter comprised between 100 nm and 1000 nm, preferably between 120 and 800 nm, more preferably between 150 and 400 nm, and a polydispersity lower than 0.25, preferably lower than 0.20, more preferably lower than 0.15, even more preferably lower than 0.10.

According to the disclosed microfluidic method, it is possible to obtain an aqueous suspension of calibrated nanodroplets by a single passage of the liquid phases through the two-channel microfluidic system.

According to a preferred embodiment, said organic phase comprises an amphiphilic lipid compound comprising a phospholipid and a fluorinated compound.

Preferably said fluorinated compound is a perfluorocarbon.

Said fluorinated compound are those described above in the present description.

In an embodiment said amphiphilic lipid compound comprising a phospholipid is a mixture of amphiphilic lipid compounds.

Still more preferably said mixture further comprises a lipid bearing polymer, being preferred a pegylated phospholipid, as defined above.

According to a further embodiment, said mixture further comprises a fatty acid, preferred being palmitic acid.

Said amphiphilic lipid compounds are those described above in the present description.

### Step A) Preparing an Aqueous Phase

The expression “aqueous phase” refers to a liquid comprising an aqueous liquid component, including for instance water, aqueous buffered solutions, aqueous isotonic solutions or a mixture thereof. Preferably the aqueous phase is water.

According to an alternative embodiment, said aqueous phase comprises an amphiphilic lipid compound as above defined.

Examples of suitable amphiphilic lipid compounds to be solubilized in the aqueous phase are pegylated phospholipids.

For instance, an amphiphilic lipid compound can be admixed with an aqueous component through traditional techniques (e.g. stirring) in order to prepare the aqueous phase to be injected into the first inlet of the microfluidic cartridge.

Said aqueous phase can further comprise an additional amphiphilic component, for instance polyoxyethylene derivatives, such as polyoxyethylene sorbitan esters (i.e. polysorbates) or polyoxyethylene-polyoxypropylene block copolymers (i.e. poloxamers).

Suitable examples of polyoxyethylene sorbitan esters are polyoxyethylene sorbitan laurate, palmitate, stearate or oleates, such as polysorbate 20 (monolaurate), polysorbate 40 (monopalmitate), polysorbate 60 (monostearate), polysorbate 65 (tristearate) or polysorbate 80 (oleate).

A suitable example of polyoxyethylene-polyoxypropylene block copolymers is Pluronic F68.

### Step B) Preparing an Organic Phase

The expression “organic phase” refers to an organic solution comprising an organic solvent miscible with water including methanol, ethanol, propanol-1, isopropanol, acetonitrile and acetone. Preferably the organic phase is ethanol.

In the present invention the expression “organic solvent miscible with water” indicates an organic solvent capable of mixing in any ratio (e.g. any concentration) with water without separation of the two phases, i.e. forming a homogeneous solution.

In a preferred embodiment, said organic phase comprises an amphiphilic lipid compound comprising a phospholipid. For instance, an amphiphilic lipid compound can be admixed with an organic solvent through traditional techniques (e.g. stirring) in order to prepare the organic solution to be mixed with the fluorinated compound.

In an embodiment, said amphiphilic lipid compound comprising a phospholipid is selected from lipid bearing polymers, fatty acids or a mixture thereof.

In a preferred embodiment said amphiphilic lipid compound comprising a phospholipid is a mixture of amphiphilic lipid compounds.

Still more preferably said mixture further comprises a lipid bearing polymer, being preferred a pegylated phospholipid, as defined above.

According to a further embodiment, said mixture further comprises a fatty acid, preferred being palmitic acid.

In an embodiment, the concentration into the organic phase of said mixture is higher than 0.5 mg/mL, preferably at least 2 mg/mL or higher preferably 4 mg/mL or higher, more preferably higher than 5 mg/mL, up to e.g. 10 mg/mL, preferably up to 8 mg/mL.

As disclosed herein, said organic phase further comprises a fluorinated compound having a boiling point comprised between −70° C. and 160° C., preferably comprised between −50° C. and 100° C., more preferably comprised between −40° C. and 60° C.

Preferably the fluorinated compound is a perfluorocarbon.

Suitable examples of fluorinated compounds are those mentioned above.

According to the disclosed method, at step B) the preparation of the organic phase to be injected into the second inlet of the microfluidic cartridge can be performed through alternative approaches depending on the nature of the fluorinated compound.

Liquid fluorinated compounds can be admixed with an organic solvent through traditional techniques (e.g. stirring) in order to prepare the organic solution.

Gaseous fluorinated compounds (having a boiling point comprised between −70° C. and 25° C. at atmospheric pressure) may be added to the organic solvent as a liquid (e.g. by dissolution into the organic solvent) or as a gas (e.g. by bubbling into the organic solvent).

### Admixing of Fluorinated Compound Having a Boiling Point Below Room Temperature (i.e. 25° C.) as Liquid Phase

According to an embodiment, step B) comprises preparing an organic phase by the addition of a fluorinated compound having a boiling point comprised between −70° C. and 25° C. at atmospheric pressure as a liquid into an organic solvent.

In an embodiment, said step B) relates to a method for the preparation of an organic phase comprising a fluorinated compound having a boiling point comprised between −70° C. and 25° C. at atmospheric pressure, said method comprising the steps of:
B.1) Condensing said fluorinated compound to obtain a liquid fluorinated compound;
B.2) Preparing a liquid organic solution comprising an organic solvent;
B.3) Cooling said liquid organic solution to a temperature below the boiling point of said liquid fluorinated compound;
B.4) Mixing said liquid fluorinated compound and said liquid organic solution to obtain an organic phase comprising a liquid fluorinated compound.

In a preferred embodiment, step B.1) is performed by cooling said fluorinated compound down to a temperature below the boiling point of said low boiling fluorinated compound.

In a further embodiment, at step B.4) the temperature at which the mixing is performed is a temperature below the boiling point of said low boiling fluorinated compound.

Preferably, the temperature at step B.4) is the same temperature of step B.3).

Still more preferably the temperature at step B.1), step B.3) and step B.4) is suitable to avoid or substantially limit the evaporation of said liquid fluorinated compound having a boiling point comprised between −70° C. and 25° C. at atmospheric pressure until to the injection of said organic phase into the microfluidic cartridge (i.e. step C).

The temperature at step B.1), step B.3) and step B.4) is lower than the boiling point of said fluorinated compound, preferably it is at least 5° C. lower than the boiling point of said fluorinated compound, more preferably at least 10° C. lower, even more preferably at least 20° C. lower, up to e.g. 50° C. lower.

The temperature of the obtained organic phase at the end of step B.4) is thus lower than the boiling point of said fluorinated compound, preferably it is at least 5° C. lower than the boiling point of said fluorinated compound, more preferably at least 10° C. lower, even more preferably at least 20° C. lower, up to e.g. 50° C. lower.

At step B.4) said mixing is performed for a time suitable to allow the dissolution of said liquid fluorinated compound into said liquid organic solution, e.g. within 5 minutes. At the end of said mixing, an organic phase is obtained in the form of a homogenous solution. Said organic phase shall be injected into the microfluidic cartridge within a time suitable for avoiding or substantially limiting the evaporation of the fluorinated compound from the organic phase, e.g. within 5 minutes from the end of step B).

In a further embodiment, at step B.4) the organic phase comprises a fluorinated compound at a concentration ranging between 1 and 100 μL/mL, preferably between 10 and 50 μL/mL, still more preferably between 15 and 30 μL/mL.

### Admixing a Fluorinated Compound Having a Boiling Point Below Room Temperature (i.e. 25° C.) as a Gaseous Phase

According to an alternative embodiment, step B) comprises preparing an organic phase by the addition of a fluorinated compound having a boiling point comprised between −70° C. and 25° C. at atmospheric pressure as gaseous phase (i.e in gas form) into an organic solvent.

In an embodiment, said step B) relates to a method for the preparation of an organic phase comprising a fluorinated compound having a boiling point comprised between −70° C. and 25° C. at atmospheric pressure, said method comprising the steps of:
B.i) Preparing a liquid organic solution comprising an organic solvent, and
B.ii) Admixing a gaseous fluorinated compound having a boiling point comprised between −70° C. and 25° C. at atmospheric pressure to said organic liquid solution.

Preferably at step B.ii) the admixing is performed by bubbling said gaseous fluorinated compound into the organic solution.

According to a preferred embodiment, before the admixing of step B.ii) the temperature of the organic solution is set above the boiling point of said gaseous fluorinated compound. A temperature above the boiling point of the fluorinated compound is generally preferred in order to avoid or substantially limit the fluorinated compound phase-shift from gas to liquid, which could lead to solubility issue of the condensed fluorinated compound into the organic solvent (e.g. formation of droplets having a liquid core filled with a fluorinated compound suspended in the organic solvent).

The temperature of the obtained organic phase at the end of step B.ii) is above the boiling point of said gaseous fluorinated compound.

Preferably, said temperature is between the boiling point and RT, more preferably up to 15° C. higher than the boiling point, even more preferably up to 10° C. higher.

At step B.ii) the concentration of said fluorinated compound into the organic phase can be any concentration, preferably up to the saturation concentration. Preferably the concentration of said fluorinated compound is the saturation concentration.

The saturation concentration of a fluorinated compound depends on the solvent used and on the temperature. For instance, considering ethanol as solvent, the saturation concentration of perfluorobutane is 2.5% by volume and the saturation concentration of perfluoropropane is 2.7% by volume (US2019307908A1).

Preferably, at step B.ii) said admixing is performed for a time suitable to reach the saturation concentration of the gaseous fluorinated compound into the organic liquid solution, e.g. for 2 minutes.

At the end of the admixing, an organic phase consisting of a homogenous solution is obtained.

In a further embodiment, at the end of its preparation process, said organic phase can be diluted by adding a suitable amount of organic solvent in order to reduce the concentration of the nanodroplets into the organic phase.

The expression “suitable amount of organic solvent” indicates the quantity of organic solvent (e.g. in mL) necessary to reduce the initial concentration of nanodroplets into the organic phase (e.g. the saturation concentration). Said organic phase is then injected into the microfluidic cartridge.

Step C) Injection into the Microfluidic Cartridge

Typically, at step C) the injection of the aqueous phase and the injection of the organic phase are carried out simultaneously.

The expression “simultaneously” indicates the simultaneous injection (i.e. co-injection) of the aqueous phase and the organic phase into the microfluidic cartridge, that is to say that the aqueous phase and organic phase are injected into two separate inlets of the microfluidic cartridge at the same time or at substantially the same time (e.g. within few seconds).

In a preferred embodiment, both aqueous and organic phases are injected into the microfluidic cartridge at a temperature suitable to avoid or substantially limit the evaporation of the fluorinated compound. For instance, after their respective preparations (i.e. step A) and step B)) both the aqueous phase and the organic phase can be stored in an ice bath (about 4° C.) before their injection into the separate inlets of the microfluidic cartridge (e.g. for 5 minutes), in order to limit the temperature increase during the time between step a), step b) and the subsequent step C).

According to the present invention, after their injections, the aqueous phase and the organic phase are directed towards a mixing device, wherein they are mixed (e.g. through laminar mixing in the case of a staggered herringbone micromixer (see FIG. 2)) endowing to the formation of NDs.

Typically, the operating pressure into the microfluidic cartridge is lower than 1000 psi (about 7000 kPa), preferably lower than 500 psi (about 3500 kPa), still more preferably lower than 300 psi (about 2000 kPa), still more preferably lower than 100 psi (about 700 kPa), e.g. between 10 and 90 psi.

The temperature of the mixing portion, wherein the mixing process takes place into the peculiar micro-channel geometry of the mixing portion, can be comprised between 0° C. and 25° C., preferably comprised between 0° C. and 15° C., more preferably between 0° C. and 5° C.

For instance, the microfluidic cartridge can be stored in the fridge (e.g. 4° C.), for a suitable time able to reach the desired temperature.

### Total Flow Rate (TFR) and Flow Rate Ratio (FRR)

The method for the present invention allows to control the nanodroplets characteristics by varying two process parameters: the Total Flow Rate and the Flow Rate Ratio.

The expression “Total Flow Rate (TFR)” refers to the total flow of both fluid streams, namely the aqueous phase and the organic phase, being pumped through the two separate inlets of the microfluidic cartridge. The unit of measurement of the TFR is mL/min.

According to an embodiment, the TFR is preferably comprised between 2 mL/min and 200 mL/min, preferably 2 and 18 mL/min, more preferably between 5 mL/min and 16 mL/min, still more preferably the TFR is 10 mL/min.

The expression “Flow Rate Ratio (FRR)” refers to the ratio between the amount of aqueous phase and the amount of organic phase flowing into the microfluidic cartridge, according to the Equation 1:

$$Flow rate ratio = \frac{volume of aqueous phase}{volume of organic phase} \qquad Eq . 1$$

The volume of aqueous and organic phases can be expressed as e.g. mL.

In a preferred embodiment, the FRR (volume of aqueous phase vs. volume of organic phase) is between 1:1 to 5:1, preferably between 1:1 and 3:1.

### Step D) Collection of the Aqueous Suspension of Calibrated Nanodroplets

According to the step D) of the disclosed method, at the end of the mixing process, an aqueous suspension of calibrated nanodroplets comprising an inner core and an outer layer, wherein said inner core comprises a fluorinated compound in liquid form and said outer layer comprises an amphiphilic lipid compound comprising a phospholipid, is collected from the exit channel of the microfluidic cartridge.

After their collection, the inner core of said microfluidically prepared nanodroplets is composed by a fluorinated compound in a liquid state independently from the temperature at which they are collected and subsequently stored and independently from the nature of fluorinated compound added to the organic phase (i.e. gaseous or liquid at SATP conditions).

In other words, even if the collection step is performed (and the subsequent storage is effected) at a temperature above the boiling point of the fluorinated compound used for the manufacturing of the calibrated NDs, said fluorinated compound remains in a liquid state due to its incorporation into the outer stabilizing shell comprising an amphiphilic component. Nevertheless, it is preferable that such temperature is not excessively high, in order to limit possible partial phase transition of the entrapped fluorinated compound.

According to an embodiment, the temperature at which said collection step is performed can be comprised between 0° C. and 25° C., preferably comprised between 0° C. and 15° C., more preferably between 0° C. and 5° C.

Preferably, the collected aqueous suspension of calibrated nanodroplets has a temperature comprised between 0° C. and 25° C., preferably comprised between 0° C. and 15° C., more preferably between 0° C. and 5° C.

For instance, after the collection said aqueous suspension can be stored at 4° C. in a fridge.

### Step E) Dilution

Step E) comprises diluting the collected aqueous suspension of calibrated nanodroplets.

As indicated above, the expressions “initial monodispersed distribution” and “initial NDs sizes” refer to the values of monodispersity and NDs sizes of the calibrated NDs composition at the end of its preparation process, wherein said end of the preparation process refers either to i) the collection of the calibrated NDs from the exit channel of the microfluidic cartridge or ii) the collection of said calibrated NDs followed by a dilution step (i.e. step E).

In the present description and claims the term “dilution” refers to the process of reducing the concentration of calibrated nanodroplets in the suspension, by adding a suitable amount of water or of an aqueous solution.

Suitable examples of aqueous solutions are saline (which may advantageously be balanced so that the final product for injection is not hypotonic), or solutions of one or more tonicity adjusting substances.

A suitable amount of water or of an aqueous solution corresponds to the quantity of aqueous solution necessary to reduce the concentration of the calibrated nanodroplets in the aqueous suspension from 2 to 10-folds.

In a preferred embodiment, the step E) of the present method comprises diluting the collected aqueous suspension of calibrated nanodroplets from 1 to 20-folds, preferably from 3 to 8-folds, still more preferably the collected aqueous suspension is diluted 5-fold.

An additional effect of dilution is that of reducing the relative amount of organic solvent in the suspension thereby reducing the sizes of the nanodroplets.

Preferably, the step E) is performed at a temperature suitable to obtain a diluted aqueous suspension of calibrated nanodroplets having a temperature comprised between 0° C. and 25° C., preferably comprised between 0° C. and 15° C., more preferably between 0° C. and 5° C.

The diluting step can be alternatively performed inside the microfluidic cartridge, by means of an additional channel (e.g. placed between the mixing device and the exit channel) aimed at diluting the calibrated fluorocarbon NDs suspension before their direction to the exit channel.

In this case, the step E) of the present method comprises diluting the suspension of calibrated fluorocarbon-filled nanodroplets from 1 to 20-folds, preferably from 3 to 8-folds, still more preferably the collected aqueous suspension is diluted 5-fold, before their collection from the microfluidic cartridge.

### Optional Step F) Washing

After the dilution step, the calibrated nanodroplets may be optionally treated using suitable washing techniques.

In the present description, the term “separation technique” indicates any operation carried out on a freshly prepared suspension of nanodroplets, finalized to remove (or substantially reduce the amount of) the organic solvent (e.g. ethanol) used to prepare said nanodroplets and by removing (or substantially reducing the amount of) not-assembled amphiphilic lipid compounds.

In the present description, the expression “not-assembled amphiphilic lipid compound” indicates amphiphilic lipid molecules that, at the end of the microfluidic preparation process, are present in the calibrated nanodroplets suspension, but are not forming the stabilizing shell of said nanodroplets.

Suitable separation techniques comprise, for instance, centrifugation and filtration (e.g. tangential flow filtration), being preferred the centrifugation.

Centrifugation parameters, such as duration, temperature and rotation may vary on the basis of the initial characteristics of the nanodroplets.

For instance, the separation phase can be carried out sequentially to the dilution phase (step E) of the disclosed microfluidic method e.g. within 5 minutes.

At the end of the separation procedure, two separate phases are obtained: a liquid supernatant phase comprising the organic solvent and a solid phase comprising the freshly prepared calibrated nanodroplets.

According to this invention, said solid phase is then admixed to an aqueous solution comprising a freeze-drying protecting component, as defined above.

According to the present invention, after obtaining a freshly-prepared suspension of nanodroplets by using any suitable method for preparation, as detailed above, said freshly prepared nanodroplets are then admixed to a freeze-drying protecting component in order to obtaining the initial suspension of step a) of the disclosed method.

Said freeze-drying protecting component is added to the suspension of freshly prepared nanodroplets in the form of a suitable aqueous solution prior to the freeze-drying (i.e. step b)).

A “suitable aqueous solution” indicates an aqueous solution of freeze-drying protecting component which, upon being admixed to the suspension of freshly prepared nanodroplets, provides an initial suspension of nanodroplets comprising a freeze-drying protecting component having a concentration comprised between 1 and 50%, preferably between 5 and 20% and still more preferably the total concentration is comprised between 8 and 12% (w/v %).

The concentration of freeze-drying protecting component in said suitable aqueous solution may thus be adjusted before the admixing to the freshly prepared suspension of nanodroplets depending for instance on the initial volume of said suspension, aiming at obtaining a specific concentration of freeze-drying protecting component after the admixing procedure.

In a preferred embodiment of the invention, before the freeze-drying process, the initial suspension of nanodroplets comprises a freeze-drying protecting component at a concentration between 1 and 50%, preferably between 5 and 20% and still more preferably the total concentration is comprised between 8 and 12% (w/v %).

The freeze-drying protecting component represents the larger amount of the final freeze-dried composition, wherein it is typically at least 75%, preferably higher than 80%, more preferably higher than 85%, up to e.g. 99.5% (w/w).

After the freeze-drying procedure, the freeze-dried composition is reconstituted with a pharmaceutically acceptable liquid carrier to obtain a reconstituted suspension of nanodroplets. The concentration of freeze-drying protecting component in said reconstituted suspension may vary depending on the volume of pharmaceutically acceptable liquid carrier used in the reconstituting phase.

Said reconstitution with a pharmaceutically acceptable liquid carrier does not comprise an additional instillation of fluorinated compound.

Suitable pharmaceutically acceptable (aqueous) liquid carrier may be water, typically sterile, pyrogen free water (to prevent as much as possible contamination in the final reconstituted product), aqueous solutions such as saline (which may advantageously be balanced so that the final product for injection is not hypotonic), or aqueous solutions of one or more tonicity adjusting substances such as salts or sugars, sugar alcohols, glycols or other non-ionic polyol materials.

The freeze-dried composition is typically reconstituted with a volume of liquid carrier similar to the volume of suspension which underwent the freeze-drying process.

Accordingly, the concentration of the freeze-drying protecting components in the reconstituted suspension is substantially the same as the one in the initial suspension.

Alternatively, the volume of said pharmaceutically acceptable liquid carrier is higher than the volume of the initial suspension of nanodroplets. In this latter embodiment, the concentration of freeze-dried protecting component in the reconstituted suspension of nanodroplets results to be lower than that comprised in the initial suspension of fluorocarbon-filled nanodroplets prior to the freeze-drying process. For instance, the volume of reconstituting liquid carrier can be twice the volume of the initial suspension, leading to a reconstituted suspension characterized by a halved concentration of freeze-drying protecting component.

Said freeze-drying protecting components are those described above in the present description.

According to an embodiment, the freeze-drying protecting component comprised in said initial suspension of nanodroplets comprises a saccharide, preferably comprises a disaccharide selected from trehalose, sucrose, maltose or a mixture thereof, more preferably said freeze-drying protecting component is trehalose.

According to another embodiment, the amphiphilic lipid compound comprising a phospholipid comprised in said initial suspension of nanodroplets is a mixture of amphiphilic lipid compounds.

Still more preferably said mixture further comprises a lipid bearing polymer, being preferred a pegylated phospholipid, as defined above.

According to a further embodiment, said mixture further comprises a fatty acid, preferred being palmitic acid.

According to an embodiment, the fluorinated compound comprised in said initial suspension of nanodroplets has a boiling point comprised between −70° C. and 160° C., preferably comprised between −50° C. and 100° C., more preferably comprised between −40° C. and 60° C.

In a preferred embodiment, said fluorinated compound is a perfluorocarbon, being selected from perfluorohexane, perfluoropentane, perfluorobutane, perfluoropropane or a mixture thereof.

### Step b) Freeze-Drying Said Initial Suspension of Nanodroplets

In this description and claims, the term freeze-drying has its standard meaning in the pharmaceutical technology field. Freeze-drying process consists of drying a pre-frozen liquid product under low pressure or vacuum and at low temperature. The main objective is to remove liquid from the product in order to provide a freeze-dried product suitable for long term storage.

Freeze-drying parameters may be selected according to the prior art, for instance the freezing temperature may range between −30° C. and −70° C. and the negative pressure applied during the freeze-drying process may be of 0.5 mbar or lower.

The freeze-drying protecting component represents the larger amount of the final freeze-dried preparation, wherein it is typically at least 75%, preferably higher than 80%, more preferably higher than 85%, up to 99.5% (w/w).

In a further aspect, the invention relates to a method for preparing a reconstituted suspension of nanodroplets, said nanodroplets comprising an inner core and an outer layer, wherein said inner core comprises a fluorinated compound in liquid form and said outer layer comprises an amphiphilic lipid compound comprising a phospholipid, comprising the steps of:
a) preparing an initial suspension comprising
i) a plurality of nanodroplets comprising an amphiphilic lipid compound comprising a phospholipid and a fluorinated compound in liquid form and
ii) a freeze-drying protecting component;
b) freeze-drying said initial suspension to obtain a freeze-drying composition, and
c) reconstituting said freeze-dried composition with a pharmaceutically acceptable liquid carrier providing a suspension of nanodroplets wherein the amount of said fluorinated compound is from 50% to 100% of the amount of fluorinated compound comprised in the initial suspension of step a).

At step c) the reconstitution of said free-drying composition does not comprise an additional instillation of fluorinated compound.

### Stability

In the present description and claims, the term “stability” refers to the property of said reconstituted suspension of nanodroplets to substantially maintain over time its initial characteristics, such as NDs sizes, size distribution, nanodroplets concentration and amount of fluorinated compound.

Particularly initial NDs sizes, initial size distribution and initial amount of fluorinated compound refer to the values of NDs sizes, size distribution and amount of fluorinated compound characterizing the initial suspension of nanodroplets as described above.

In other words, said initial characteristics are those possessed by the initial suspension of nanodroplets before its freeze-drying and subsequent storage period.

The expression “storage period” indicates a period of time, e.g. expressed as hours, days or weeks, during which the freeze-dried composition is kept under certain conditions (e.g. of temperature) after the end of the preparation process.

According to an embodiment, said freeze-dried composition is stored at a temperature not higher than 30° C., preferably not higher than 25° C., more preferably not higher than 10° C., still more preferably said temperature is 4° C. or less, up to −20° C.

According to the present invention it is possible to improve the stability of a reconstituted suspension of nanodroplets by performing the freeze-drying in the presence of a suitable freeze-drying protecting component.

After the freeze-drying, the reconstituted suspensions of nanodroplets were found to be characterized by substantially the same initial characteristics of the initial suspension. In particular, said initial characteristics resulted to be essentially preserved after the freeze-dried procedure, even after storing the freeze-dried composition for 3 months at 4° C. On the contrary, initial suspensions of nanodroplets comprising a freeze-dried protecting component, not submitted to a freeze-drying process, were not able to maintain their sizes, size distribution and amount of fluorinated compound over time.

In an embodiment said freeze-dried composition upon reconstitution with a pharmaceutically acceptable liquid carrier provides a suspension of nanodroplets, wherein the amount of said fluorinated compound is from 50% to 100% of the amount of fluorinated compound comprised in the initial suspension, preferably from 60% to 100%, more preferably from 70 to 100%, still more preferably from 80 to 100% of the amount of fluorinated compound comprised in the initial suspension of step a).

The Applicant has unexpectedly observed that it is possible to substantially preserve the amount of fluorinated compound comprised in the initial suspension of nanodroplets after the freeze-drying process without any additional instillation of fluorinated compound.

Surprisingly, the reconstituted suspensions of nanodroplets substantially maintain the initial amount of fluorinated compound as characterized before the freeze-drying process, and thus suitable for subsequent pharmaceutical uses without any further addition of fluorinated compound (e.g. neither comprised in the pharmaceutically acceptable liquid carrier nor instilled as a gas to in the freeze-dried product before the reconstitution step).

Substantially preserving the amount of fluorinated compound advantageously endows to a reconstituted suspension of nanodroplets having a similar acoustic response as the initial suspension prior to freeze-drying. In other words, the reconstituted suspension of nanodroplets may be converted into gas-filled bubbles upon exposure to a substantially similar acoustic pressure in comparison to that necessary to convert the suspension before the freeze-drying process.

The amount of fluorinated compound (FC) in the reconstituted suspension of nanodroplets can be expressed as a percentage of the initial amount of fluorinated compound in the initial suspension of nanodroplets by using the following equation (Equation 2):

$$\% FC in the reconstituted suspension = \frac{amount of FC after freeze - drying}{amount of FC before freeze - drying} * 100 \qquad Eq . 2$$

Wherein:
the expression “amount of fluorinated compound after freeze-drying” refers to the weight (e.g. mg) of fluorinated compound comprised in 1 ml of reconstituted suspension of nanodroplets comprised in the vial after freeze-drying and
the expression “amount of fluorinated compound before freeze-drying” refers to the weight (e.g. mg) of fluorinated compound comprised in 1 ml of initial suspension of nanodroplets comprised in the vial before freeze-drying.

Said amount can be measured for instance by analytical methods such gas chromatography (GC).

A good preservation of the nanodroplets characteristics in terms of sizes and size distribution was associated with a % amount of fluorinated compound in the reconstituted suspension higher than 50% of the amount of fluorinated compound in the initial suspension, preferably higher than 60%, more preferably higher than 70%, still more preferably higher than 80%, up to 100%.

The Applicant found that it is possible to obtain said amount of fluorinated compound in the reconstituted suspension by admixing a suitable freeze-drying protecting component to the freshly prepared nanodroplets prior to the freeze-drying process, and without any further addition of fluorinated compound to the freeze-dried composition, before its reconstitution with a pharmaceutical liquid carrier.

The Applicant demonstrated in particular that the reconstituted suspensions of nanodroplets comprising a disaccharide as freeze-drying protecting component maintained unexpectedly high amount of fluorinated compound, up to 100% of the amount of fluorinated compound in the initial suspension.

Another aspect relates to a reconstituted suspension of nanodroplets prepared by reconstituting a freeze-dried composition with a pharmaceutically acceptable liquid carrier, wherein said freeze-dried composition is obtained by freeze drying an initial suspension comprising
i) a plurality of nanodroplets comprising an amphiphilic lipid compound comprising a phospholipid and a fluorinated compound in liquid form and
ii) a freeze-drying protecting component;
and wherein the amount of said fluorinated compound is from 50% to 100% of the amount of fluorinated compound in liquid form comprised in the initial suspension prior to freeze-drying.

### Weight Ratio Freeze-Drying Protecting Component/Fluorinated Compound

In the present description and claims the expression “weight ratio between freeze-drying protecting component and fluorinated compound” indicates the ratio between the weight amount (e.g. mg) of freeze-drying protecting component (FDPC) comprised in a given volume (e.g. mL) of suspension of nanodroplets and the weight amount (e.g. mg) of fluorinated compound comprised in the same given volume (e.g. mL) of suspension of nanodroplets. Said “weight ratio” corresponds to the ratio used to stabilize the disclosed nanodroplets. It is possible to calculate the weight ratio by using the following equation (Equation 3):

$$Weight ratio FDPC / FC = \frac{total weight of freeze - drying protecting component}{total weight of FC} \qquad Eq . 3$$

wherein:
the expression “total weight of freeze-drying protecting component” indicates the weight, express in mg, of freeze-drying protecting component comprised in 1 ml of initial suspension of nanodroplets prior to the freeze-drying, and
the expression “total weight of (P)FC” indicates the weight amount of fluorinated compound comprised in 1 ml of initial suspension of nanodroplets prior to the freeze-drying.

In an embodiment, in said initial suspension of nanodroplets prior to freeze-drying the weight ratio between said freeze-drying protecting component and said fluorinated compound is higher than 10, preferably higher than 20, preferably higher than 30, preferably higher than 50, preferably higher than 60, preferably higher than 100, up to 250.

According to a preferred embodiment the weight ratio between said freeze-drying protecting component and said fluorinated compound freeze-drying protecting component and fluorinated compound is substantially unvaried after the freeze-drying process.

Thus, according to said embodiment in the freeze-dried composition the weight ratio between said freeze-drying protecting component and said fluorinated compound is higher than 10, preferably higher than 20, preferably higher than 30, preferably higher than 50, preferably higher than 60, preferably higher than 100, up to 250.

Said weight ratio is also essentially the same in the reconstituted suspension of nanodroplets.

According to an embodiment, in said reconstituted suspension composition the weight ratio between said freeze-drying protecting component and said fluorinated compound is higher than 10, preferably higher than 20, preferably higher than 30, preferably higher than 50, preferably higher than 60, preferably higher than 100, up to 250.

### Use

The acoustic droplet vaporization (ADV) is a phenomenon through which nanodroplets with a liquid inner core comprising a fluorinated compound can be converted into gas-filled microbubbles upon exposure to ultrasound energy beyond the vaporization threshold.

When administered in-vivo, nanodroplets present many advantages with respect to traditional microbubbles, such as inertness, relatively low toxicity, relative stability in circulation, immiscibility in water, and low surface tension. Once vaporized, the generated microbubbles can be effectively used in either imaging or therapeutic applications with ultrasound, including sonopermeabilization, thermal ablation, blood brain barrier (BBB) disruption, multimodal imaging modalities and allow passive (due to the enhanced permeability and retention (EPR) effect in the tumor tissues) or active targeting (by incorporating targeted ligands) for localized delivery of therapeutic drugs or genes. Another potentially valuable characteristic of (P)FC-NDs is their possible application for novel imaging strategies such as UltraSound Super-Resolution Imaging since these agents can be activated and deactivated on demand by applying intermittent acoustic pulses.

A further aspect relates to a freeze-dried composition comprising
i) an amphiphilic lipid compound comprising a phospholipid,
ii) a fluorinated compound in liquid form and
iii) a freeze-drying protecting component
which, upon reconstitution with a pharmaceutically acceptable liquid carrier, provides a suspension of nanodroplets, said nanodroplets comprising an inner core and an outer layer, wherein said inner core comprises a fluorinated compound and said outer layer comprises amphiphilic lipid compound for use in a diagnostic and/or therapeutic treatment.

Diagnostic treatment includes any method where the use of the nanodroplets allows enhancing the visualization of a portion or of a part of an animal (including humans) body, including imaging for preclinical and clinical research. Suitable examples of diagnostic applications are molecular and perfusion imaging, tumor imaging (EPR effect), multimodal imaging (MR-guided tumor ablation, fluorescence, sono-photoacoustic activation), US aberration correction and super-resolution imaging.

Therapeutic treatment includes any method for treatment of a patient. In preferred embodiments, the treatment comprises the combined use of ultrasounds and nanodroplets either as such (e.g. in ultrasound-mediated thrombolysis, high intensity focused ultrasound ablation, blood-brain barrier permeabilization, immunomodulation, neuromodulation, radiosensitization) or in combination with a therapeutic agent (i.e. ultrasound-mediated delivery, e.g. for the delivery of a drug or bioactive compound to a selected site or tissue, such as in tumor treatment, gene therapy, infectious diseases therapy, metabolic diseases therapy, chronic diseases therapy, degenerative diseases therapy, inflammatory diseases therapy, immunologic or autoimmune diseases therapy or in the use as vaccine), whereby the presence of the nanodroplets may provide a therapeutic effect itself or is capable of enhancing the therapeutic effects of the applied ultrasounds, e.g. by exerting or being responsible to exert a biological effect in vitro and/or in vivo, either by itself or upon specific activation by various physical methods (including e.g. ultrasound-mediated delivery).

The following examples will help to further illustrate the invention.

### EXAMPLES

The following materials are employed in the subsequent examples:

TABLE 1
Materials used in the subsequent Examples

| | | | Formula weight |
|---|---|---|---|
| Material | | Molecular formula | (g/mol) |
| DPPC | 1,2-dipalmitoyl-sn-glycero-3- | C₄₀H₈₀NO₈P | 734 |
| | phosphocholine | | |
| DPPE-PEG5000 | 1,2-dipalmitoyl-sn-glycero-3- | C₂₆₅H₅₃₁N₂O₁₂₃P | 5745 |
| | phosphoethanolamine-N- | | |
| | [methoxy(polyethylene glycol)- | | |
| | 5000] (ammonium salt) | | |
| DMPA | 1,2-dimyristoyl-sn-glycero-3- | C₃₁H₆₀O₈PNa | 614 |
| | phosphate (sodium salt) | | |
| PA | Palmitic acid | C₁₆H₃₂O₂ | 256 |
| C₆F₁₄ | Perfluorohexane | C₆F₁₄ | 338 |
| C₅F₁₂ | Perfluoropentane | C₅F₁₂ | 288 |
| C₄F₁₀ | Perfluorobutane | C₄F₁₀ | 238 |
| C₃F₈ | Perfluoropropane | C₃F₈ | 188 |
| Ethanol | Ethyl alcohol | C₂H₅OH | 46 |
| Trehalose | | C₁₂H₂₂O₁₁ | 342.3 |
| Sucrose | | C₁₂H₂₂O₁₁ | 342.3 |
| Mannitol | | C₆H₁₄O₆ | 182.2 |
| Maltose | | C₁₂H₂₂O₁₁ | 342.3 |
| Dextran 15 | | | 15′000 |
| PEG4000 | Polyethylene glycol 4000 | | 4′000 |

### Example 1

### Preparation of Nanodroplets Using a Microfluidic Platform

Calibrated nanodroplets were formulated with a NanoAssemblr™ Benchtop automated instrument from Precision Nanosystems (Vancouver, Canada) equipped with a staggered herringbone micromixer (SHM) (NxGen Cartridge, without in-line dilution) allowing size-controlled self-assemblies.

### 1.1. Organic Phase Comprising a High Boiling Point Fluorinated Compound

An organic solution was prepared by adding a lipid mixture of DPPC/PA/DPPE-PEG5k (with a molar ratio of 74.1/18.5/7.4) at 6 mg/mL in ethanol and it was cooled down to about 4° C. for at least 15 minutes. An organic phase was thus obtained by adding a high boiling point perfluorocarbon (e.g. perfluoropentane (C₅F₁₂, b.p. 29.2° C.) or perfluorohexane (C₆F₁₄, b.p. 57.1° C.)) into the lipid mixture at a concentration of 10 μL/mL.

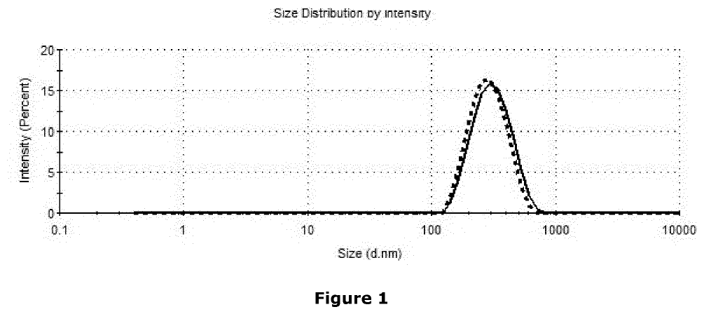

An aqueous phase composed of MilliQ water (at 4° C.) was injected into the first inlet whereas the organic phase was injected into the second inlet of the microfluidic cartridge (FIG. 1). Microscopic characteristics of the channels are engineered to cause an accelerated mixing of the two fluid streams in a controlled fashion. The microfluidic process settings, namely the Total Flow Rate (TFR, in mL/min) and the Flow Rate Ratio (FRR), were FRR of 3-1 (Aqueous-Organic) and TFR of 10 mL/min. The NDs suspensions were collected from the exit channel in a Falcon vial (15 mL) and 5-fold diluted with ultrapure water.

### 1.2. Organic Phase Comprising a Low Boiling Point Fluorinated Compound

An organic solution was prepared by adding a lipid mixture of DPPC/PA/DPPE-PEG5k (with a molar ratio of 74.1/18.5/7.4) at 6 mg/mL in ethanol and it was cooled down to about −20° C. Then, liquid perfluorobutane (C₄F₁₀, b.p. −2° C.) was obtained by condensation of PFB gas into a syringe at −20° C. and was added to the lipid ethanolic solution at a concentration of 15 μL/mL. The solution was stirred until the total dissolution of liquid perfluorobutane. The same procedure was carried out using perfluoropropane (boiling point −37° C.) as perfluorocarbon, except that the lipid mixture in ethanol was cooled down to about −80° C. and that liquid perfluoropropane was obtained by condensation into a syringe at −80° C.

An aqueous phase composed of MilliQ water (at 4° C.) was injected into the first inlet whereas the organic phase was injected into the second inlet of the microfluidic cartridge (FIG. 1). Microscopic characteristics of the channels are engineered to cause an accelerated mixing of the two fluid streams in a controlled fashion. The microfluidic process settings, namely the Total Flow Rate (TFR, in mL/min) and the Flow Rate Ratio (FRR), were FRR of 1-1 (Aqueous-Organic) and TFR of 10 mL/min. The NDs suspensions were collected from the exit channel in a Falcon vial (15 mL) and 5-fold diluted with ultrapure water.

### Example 2

### Preparation of Perfluorocarbon-Filled Nanodroplets by Sonication Method

An organic solution was prepared by adding a lipid mixture of DPPC/PA/DPPE-PEG5k (with a molar ratio of 74.1/18.5/7.4) at 6 mg/mL in ethanol. The solution was then heated at 65° C. in a 100 mL balloon under stirring. The solvent was removed under vacuum to obtain a lipid film. The lipid blend was dried under vacuum (0.2 mBar) at 25° C. overnight.

The lipid blend was redispersed in 10 mL of Tris buffer 20 mM pH 7.5 at 65° C. under stirring for 30 min. The obtained liposomal solution was placed in an ultrasound tank for 3 minutes. The suspension was then cooled to 4° C. in an ice bath.

0.5 mL of perfluoropentane (C₅F₁₂, b.p. 29.2° C.) were added to the liposomal suspension. The mixture was emulsified with a Branson Sonifier 250 fitted with a 5 mm tip (output 20%—6×(1 min US on—30 s US off)). During all the process, the suspension was cooled in an ice bath. The ND size was measured after emulsification.

### Example 3

### Preparation of Perfluorocarbon-Filled Nanodroplets by Microbubble Condensation Method

Perfluorobutane-filled microbubbles were prepared as described in US2022211850A1 (Example 6).

After their preparation the reconstitution of lipid-shelled microbubbles (MBs) was performed by adding 5 mL of sterile saline solution (0.9% NaCl) into the vial using a syringe and mixing for about 10 seconds to obtain a white milky homogeneous liquid.

Subsequently a 10-mL syringe was used to take a 4 mL-sample of said MBs suspension (6 ml of headspace) and then stoppered and placed at about −20° C. for about 6 minutes, avoiding suspension freezing.

Then the syringe piston was pushed down up to the ˜4 mL graduation/scale to increase the pressure inside the syringe and maintained in that position for about 30 s, then the syringe piston was slowly released in order to obtain a limpid suspension of nanodroplets, characterized by a core comprising perfluorobutane in liquid state, without microbubbles. The liquid core was stable even during the subsequent storage of the suspension effected at a temperature above the boiling point of the perfluorobutane.

### Example 4

### Preparation of the Freeze-Dried Composition

The suspensions of nanodroplets obtained according to the previous Example 1, 2 and 3, were admixed with suitable solutions of freeze-drying protecting components before proceeding to the freeze-drying procedure.

For this purpose, the admixing phase was carried out following different protocols depending on the method for preparation used, as described in the following paragraphs.

22.5 mL of the calibrated nanodroplets suspension obtained through the microfluidic method, as described in Example 1, were centrifuged at 5000 g for 12 minutes at 4° C. The supernatant was discarded, and the washed nanodroplets (pellet) were redispersed with a solution comprising the freeze-drying protecting component to obtain 22.5 mL of suspension as detailed in the following examples. 1 ml of perfluorocarbon-filled nanodroplets suspension obtained by sonication method, as described in Example 2, were directly admixed to 9 ml of a solution comprising the freeze-drying protecting component.

The suspension of perfluorocarbon-filled nanodroplets obtained via microbubbles condensation method as described in Example 3, was diluted of 2-fold with a trehalose solution (20%).

The specific type and amounts of materials used in said suspensions are summarized in Table 2.

Said suspensions were then aliquoted in DIN8R glass vials (1 mL suspension/vial), transferred in the freeze dryer shelves and frozen at −55°. The freeze-drying procedure was then carried out by performing a drying step at −20° C. and 200 μBar. At the end of the freeze drying, the vials were stoppered, crimped and stored at 4° C. until redispersion.

At the end of the procedure, a freeze-dried composition was obtained as a white homogenous dry solid. The vials were stoppered, crimped, and stored at 4° C. until redispersion.

In the following examples, after the freeze-drying process, each freeze-dried composition was reconstituted with 1 mL of aqueous liquid carrier, in order to obtain a stable nanodroplets suspension. The freeze-dried composition was thus reconstituted with a volume of aqueous liquid carrier equal to the volume of suspension which underwent the freeze-drying process.

TABLE 2
Compositions used in the present invention: all the compositions comprise
DPPC/PA/DPPE-PEG5000 (m.r. 74.1/18.5/7.4) as stabilizing shell materials,
except composition 5PT comprising DSPC/PA/DPPE-PEG5000 (m.r. 74.1/18.5/7.4)

| | | Freeze-drying | | |
|---|---|---|---|---|
| | | protecting | Weight ratio Freeze- | |
| Composition | Perfluorocarbon | component | drying protecting | |
| nr | (type) | (type; conc wt %) | component/PFC | Preparation Method |
| 1T | Perfluorohexane | Trehalose 10% | 120 | MICROFLUIDIC |
| 1S | Perfluorohexane | Sucrose 10% | 120 | MICROFLUIDIC |
| 1M | Perfluorohexane | Mannitol 10% | 120 | MICROFLUIDIC |
| 1P | Perfluorohexane | PEG4000 10% | 120 | MICROFLUIDIC |
| 1D | Perfluorohexane | Dextran 15 10% | 120 | MICROFLUIDIC |
| 1Ma | Perfluorohexane | Maltose 10% | 52 | MICROFLUIDIC |
| 3T | Perfluoropentane | Trehalose 10% | 123 | MICROFLUIDIC |
| 3S | Perfluoropentane | Sucrose 10% | 123 | MICROFLUIDIC |
| 3M | Perfluoropentane | Mannitol 10% | 123 | MICROFLUIDIC |
| 1Ta | Perfluorohexane | Trehalose 5% | 60 | MICROFLUIDIC |
| 1Tb | Perfluorohexane | Trehalose 15% | 180 | MICROFLUIDIC |
| 1Tc | Perfluorohexane | Trehalose 20% | 240 | MICROFLUIDIC |
| 2Ta | Perfluoropentane | Trehalose 10% | 6.25 | SONICATION |
| 2Tb | Perfluoropentane | Trehalose 10% | 12.5 | SONICATION |
| 2Tc | Perfluoropentane | Trehalose 10% | 25 | SONICATION |
| 2Td | Perfluoropentane | Trehalose 10% | 62.5 | SONICATION |
| 4T | Perfluorobutane | Trehalose 10% | 42 | MICROFLUIDIC |
| 4S | Perfluorobutane | Sucrose 10% | 42 | MICROFLUIDIC |
| 4Ma | Perfluorobutane | Maltose 10% | 42 | MICROFLUIDIC |
| 4P | Perfluorobutane | PEG4000 5% | 267 | CONDENSATION |
| 4PT | Perfluorobutane | PEG4000 5% | 407 | CONDENSATION |
| | | Trehalose 10% | | |
| 5PT | Perfluorobutane | PEG4000 5% | 407 | CONDENSATION |
| | | Trehalose 10% | | |
| 5T | Perfluoropropane | Trehalose 10% | 44 | MICROFLUIDIC |

### Example 5

### Stability of Freeze-Dried Perfluorocarbon-Filled Nanodroplets

In order to evaluate the effect of the freeze-drying process on the NDs initial properties, different storage conditions were compared for composition 1T.

As comparison, before the freeze-drying procedure an aliquot of each sample was stored as such in the form of aqueous suspension at 4° C. over time.

The nanodroplets suspensions, reconstituted after freeze-drying or stored as such, were characterized using a Malvern Zetasizer Nano-ZS instrument (Malvern Instruments Ltd., UK) to measure the size and size distribution (PDI) after different storage conditions, as illustrated in Table 3.

### Results

The overall results displayed that freeze-drying allowed a better preservation of NDs characteristics (size and polydispersity index) overtime compared to storing the ND suspension at 4° C.

TABLE 3
Variation of sizes and PDI of the freeze-dried product in comparison
with a not-lyophilized suspension over different storage conditions

| | | Z average | |
|---|---|---|---|
| | Storage conditions | (nm) | PDI |
| Composition 1T | Before freeze drying | 213 | 0.069 |
| | After freeze drying | 206 | 0.085 |
| | 3 months at 4° C. after | 207 | 0.104 |
| | freeze-drying | | |
| | ND suspension after 1 day | 354 | 0.187 |
| | at 4° C. | | |
| | ND suspension after 3 | 378 | 0.646 |
| | months at 4° C. | | |

### Example 6

### Effects of the Nature of Freeze-Drying Protectins Components on the Nanodroplets Characteristics

Different freeze-drying protecting components were selected to evaluate the influence of their nature on the nanodroplets characteristics.

The detailed qualitative/quantitative composition of each formulation characterized in the present example are reported in Table 2. The characterization (Z average and PDI) was performed for all compositions except 4PT and 5PT using a Malvern Zetasizer Nano-ZS instrument (Malvern Instruments Ltd., UK) to measure the size and size distribution (PDI) after different storage conditions, as illustrated in Table 4.

The characterization of the Compositions 4PT and 5PT was performed using NanoSight NS300 (Malvern) equipment.

Nanodroplets concentration after different storage conditions was measured by NTA (Nanoparticles Tracking Analysis).

NTA measures were carried out with NanoSight NS300 (Malvern) equipment according to manufacturer recommendations.

### Results

The overall results showed that using disaccharides (in particular trehalose, sucrose or maltose) as freeze-drying protecting agents led to a substantial good preservation of the initial nanodroplets sizes, PDI and concentration after freeze-drying.

NDs properties were substantially maintained using disaccharides independently from the nature of the NDs core (i.e. fluorinated compound with high or low boiling point). Indeed, positive results in term of properties preservation were reached with both PFC with high boiling point, such as perfluorohexane (b.p. 57.1° C.) (Comp. 1T, 1S and 1Ma) and perfluoropentane (b.p. 29.2° C.) (Comp. 3T and 3S), or PFC with low boiling point, such perfluorobutane (b.p. −2° C.) (Comp. 4T, 4S and 4Ma) and perfluoropropane (b.p. −37° C.) (Comp. 5T).

Nanodroplets concentrations were measured by NTA on the Composition 1T and 1Ma, showing that the concentration was substantially maintained after freeze-drying (Comp. 1T: 8.64×10¹⁰ NDs/mL before freeze-drying and 7.49×10¹⁰ NDs/mL after freeze-drying; Comp-1Ma: 8.66×10¹⁰ NDs/mL before freeze-drying and 7.33×10¹⁰ NDs/mL after freeze-drying).

The results further confirmed that the use of mixtures comprising a disaccharide as freeze-drying protecting agent led to a substantially good preservation of NDs properties, e.g. sizes, after freeze-drying (see Compositions 4PT and 5PT). Both these formulations, obtained by microbubble condensation method were characterized by a substantially high polydispersity before and after freeze-drying.

Despite of a substantial good preservation of the initial NDs sizes, mannitol and PEG4000 (used as single freeze-drying protecting agent) endowed to a less efficient conservation of the initial PDI values after the freeze-drying process. Compositions comprising Dextran 15 already showed high PDI value (>0.250) after their preparation, before the freeze-drying procedure. The freeze-dried Compositions 1D were characterized by significantly different sizes and PDI after the freeze-drying procedure, either immediately after the end of the process or after a 3-month storage period.

TABLE 4
Effects of the nature of freeze-drying protecting component
on the sizes and PDI before and after freeze-drying

| | |
|---|---|
| Before Freeze Drying | After Freeze Drying |

| Composition | Z average | | Z average | |
|---|---|---|---|---|
| nr | (nm) | PDI | (nm) | PDI |
| 1T | 213 | 0.069 | 206 | 0.085 |
| 3T | 267 | 0.093 | 294 | 0.130 |
| 4T | 268 | 0.249 | 301 | 0.223 |
| 5T | 234 | 0.162 | 257 | 0.210 |
| 1S | 223 | 0.092 | 220 | 0.124 |
| 3S | 317 | 0.149 | 314 | 0.196 |
| 4S | 276 | 0.260 | 290 | 0.206 |
| 4Ma | 337 | 0.374 | 294 | 0.235 |
| 1Ma | 230 | 0.207 | 235 | 0.198 |
| 1M | 203 | 0.126 | 133 | 0.422 |
| 3M | 271 | 0.160 | 152 | 0.511 |
| 1P | 200 | 0.088 | 176 | 0.326 |
| 1D | 126 | 0.663 | 50 | 1.000 |
| 4PT | 173 | NA* | 199 | NA* |
| 5PT | 192 | NA* | 186 | NA* |

*Not Applicable due to polydisperse profile

### Example 7

Determination of the Amount of Perfluorocarbon after Freeze-Drying

The influence of the freeze-drying procedure on the amount of perfluorocarbon comprised in the reconstituted suspension of nanodroplets was evaluated. For this purpose, the content of the fluorocarbon was measured by Gas Chromatography coupled with a Flame ionization detector (GC-FID) before and after the freeze drying. The amount of perfluorocarbon in the reconstituted suspension of perfluorocarbon-filled NDs was calculated according to Equation 2.

### Results

TABLE 5
Influence of the freeze-drying protecting component
(nature and quantity) on the amount of perfluorocarbon
preserved after freeze-drying procedure

| Composition | Remaining PFC |
|---|---|
| nr | (%) |
| 3T | 100 |
| 4T | 100 |
| 3S | 82 |
| 3M | 8 |

Results demonstrated that reconstituted suspensions of PFC-NDs comprising a disaccharide as freeze-drying protecting component maintained a higher amount of perfluorocarbon than using other freeze-drying protecting components. In particular, the amount of perfluorocarbon was found to be about ten-times higher using trehalose, saccharose and maltose than the amount of perfluorocarbon of a suspension comprising mannitol.

Unexpectedly the amount of perfluorocarbon was found to be substantially unvaried, i.e. close to 100% of the amount of perfluorocarbon in the initial suspension.

The good preservation of the amount of perfluorocarbon was confirmed with both PFC with high boiling po, such as perfluoropentane (b.p. 29.2° C.) (Comp. 3T and 3S) and perfluorohexane (b.p. 57.1° C.) (Comp. 1Ma), or PFC with low boiling, such perfluorobutane (b.p. −2° C.) (Comp. 4T).

### Example 8

### Influence of the Weight Ratio Between the Freeze-Drying Protecting Component and the Perfluorocarbon

### 8.1. Calibrated PFC Nanodroplets Obtained by Microfluidic Technique

Different freeze-drying protecting component/PFC weight ratios were investigated to evaluate their influence on the nanodroplets characteristics before and after freeze-drying. For this purpose, the procedure illustrated in Example 1 and Example 4 was performed by varying the amount of freeze-drying protecting component agent added to the washed nanodroplets (pellet) after centrifugation. In the present example, the initial freeze-drying protecting component/PFC weight ratios were 60, 120, 180 and 240, obtained by using aqueous solution of trehalose at 5, 10, 15 and 20% trehalose.

The weight ratios were determined according to Equation 3, as described above.

At the end of the freeze drying, the vials were stoppered, sealed and stored at 4° C. until redispersion.

The detailed qualitative/quantitative composition of each formulation characterized in the present example, together with the process parameters used for their preparation, are reported in Table 2.

The characterization was performed using a Malvern Zetasizer Nano-ZS instrument (Malvern Instruments Ltd., UK) to measure the size and size distribution (PDI) before and after freeze-drying, as illustrated in Table 6.

### Results

The results demonstrated a substantially good preservation of the initial ND characteristics in terms of sizes (Z-average and PDI) in all the investigated Compositions having different initial freeze-drying protecting component/PFC ratios (from 60 to 240).

TABLE 6
Influence of the weight ratio between the freeze-drying protecting
component and the perfluorocarbon on the sizes and PDI

| | Freeze-drying | | | | |
|---|---|---|---|---|---|
| Compo- | protecting | Before Freeze Drying | | After Freeze Drying | |
| sition | component/ | Z average | | Z average | |
| nr | PFC ratio | (nm) | PDI | (nm) | PDI |
| 1Ta | 60 | 230 | 0.095 | 234 | 0.188 |
| 1T | 120 | 213 | 0.069 | 206 | 0.085 |
| 1Tb | 180 | 198 | 0.094 | 200 | 0.139 |
| 1Tc | 240 | 183 | 0.100 | 187 | 0.097 |

### 8.2. PFC Nanodroplets Obtained by Sonication Technique

PFC nanodroplets were prepared according to the procedures illustrated in Example 2. After their preparation, the obtained suspensions were then diluted 5-fold, 10-fold, 20-fold or 50-fold with an aqueous solution of trehalose solution (final trehalose concentration 10% by weight in the suspension), in order to obtain a corresponding freeze-drying protecting component/PFC weigh ratio of 6.25, 12.5 and 25, respectively. The obtained suspensions were sampled in DIN8R vials (1 mL/vial) and then freeze-dried according to Example 4. At the end of the freeze drying, the vials were stoppered, sealed and stored at 4° C. until redispersion.

The detailed quali-quantitative composition of each formulation characterized in the present example, together with the process parameters used for their preparation, are reported in Table 2.

The characterization was performed using a Malvern Zetasizer Nano-ZS instrument (Malvern Instruments Ltd., UK) to measure the size and size distribution (PDI) before and after freeze-drying, as illustrated in Table 7.

### Results

As inferable from Table 7, the Compositions characterized by lower weight ratios between freeze-drying protecting component/PFC underwent to an increase of the ND sizes and the PDI after the freeze-drying procedure. Compositions with higher ratios, substantially preserved both initial sizes and PDI.

TABLE 7
Influence of the weight ratio between the freeze-drying protecting
component and the perfluorocarbon on the sizes and PDI

| | Freeze-drying | Before Freeze | | After Freeze | |
|---|---|---|---|---|---|
| | protecting | Drying | | Drying | |
| Composition | component/PFC | Z average | | Z average | |
| nr | ratio | (nm) | PDI | (nm) | PDI |
| 2Ta | 6.25 | 309 | 0.191 | 336 | 0.356 |
| 2Tb | 12.5 | 309 | 0.206 | 335 | 0.279 |
| 2Tc | 25 | 286 | 0.246 | 266 | 0.239 |

### Example 9

### Determination of Acoustic Droplet Vaporization (ADV)

The expression “Acoustic Droplet Vaporization (ADV) threshold” indicates the minimal acoustic pressure that is necessary to obtain the nanodroplets conversion into echogenic gas-microbubbles.

The Acoustic Droplet Vaporization (ADV) threshold of the calibrated PFC NDs prepared according to the previous examples can be determined according to conventional methodologies using B-mode imaging methods. For instance, the suspension of NDs can be vaporized while passing through the focal zone of a transducer and the acoustic pressure is increased of about 0.2 MPa each 5 s until the NDs vaporization is observed.

Nanodroplets activation was performed by focused ultrasound waves on five aligned focal points allowing the activation only within the region of interest where the acoustics pressure was highest. Pulses were emitted in burst mode at a frequency of 6 MHz, 20 cycles per pulse and at a pulse-repetition frequency (PRF) of 1 Hz.

The ADV determination was performed on five different compositions, distinguished by the PFC forming their core, namely perfluorohexane, perfluoropentane and perfluorobutane (see Table 8). The ADV of each composition was determined before and after the freeze-drying procedure.

For all experiments, 0.4 mL of each NDs suspension were diluted in 40 mL of water at 37° C.

### Results

Table 9 reports the overall results obtained from the determination of the ADV thresholds. Each value is the average of three successive determinations.

TABLE 8
Acoustic Droplet Vaporization threshold as function of PFC core

| | Before freeze-drying | After freeze-drying |
|---|---|---|
| Composition nr | MPa | MPa |
| 1T | 10.28 | 10.33 |
| 3T | 9.14 | 9.15 |
| 4T | 4.59 | 4.35 |
| 4PT | 4.20 | 4.12 |
| 5PT | 4.50 | 4.30 |

It emerges that all the tested compositions had similar ADV threshold values before and after the freeze-drying procedure, independently on the nature of the PFC in the core of the NDs, confirming that the acoustic response was also preserved.

### REFERENCES

Zhang, W. et al, Nanoscale, 2022, 14, 2943-2965
Melich, R. et al. International Journal of Pharmaceutics, 2020, 587, 119651
US20220000790
WO2022101365A1 (Bracco Suisse SA)
US20190307908
US2022211850A1

1. A freeze-dried composition comprising
i) an amphiphilic lipid compound comprising a phospholipid,
ii) a fluorinated compound in liquid form and
iii) a freeze-drying protecting component
which, upon reconstitution with a pharmaceutically acceptable liquid carrier, is capable of providing a suspension of nanodroplets comprising an inner core and an outer layer, wherein said inner core comprises said fluorinated compound in liquid form and said outer layer comprises said amphiphilic lipid compound.

2. The freeze-dried composition according to claim 1, wherein the weight ratio between said freeze-drying protecting component and said fluorinated compound is higher than 10, up to 250.

3. The freeze-dried composition according to claim 2, wherein said weight ratio is higher than 20.

4. The freeze-dried composition according to claim 3, wherein said weight ratio is higher than 50.

5. The freeze-dried composition according to claim 1, wherein said fluorinated compound has a boiling point comprised between −70° C. and 160° C.

6. (canceled)

7. (canceled)

8. The freeze-dried composition according to claim 1, wherein said fluorinated compound is a perfluorocarbon selected from the group consisting of perfluoropentane, perfluorohexane, perfluorobutane, perfluoropropane and a mixture thereof.

9. The freeze-dried composition according to claim 1, wherein said freeze-drying protecting component is a saccharide.

10. The freeze-dried composition according to claim 9, wherein said saccharide is a disaccharide selected from the group consisting of trehalose, sucrose, maltose and a mixture thereof.

11. The freeze-dried composition according to claim 10, wherein said disaccharide is trehalose.

12. The freeze-dried composition according to claim 1, wherein the amount of said freeze-drying component is at least 75%, up to 99.5% (w/w).

13. (canceled)

14. (canceled)

15. A method for preparing a freeze-dried composition as defined in claim 1, comprising the steps of:
a) preparing an initial suspension comprising
i) a plurality of nanodroplets comprising an amphiphilic lipid compound comprising a phospholipid and a fluorinated compound in liquid form and
ii) a freeze-drying protecting component; and
b) freeze-drying said initial suspension
wherein said freeze-dried composition upon reconstitution with a pharmaceutically acceptable liquid carrier is capable of providing a suspension of nanodroplets comprising an inner core and an outer layer, wherein said inner core comprises said fluorinated compound in liquid form and said outer layer comprises said amphiphilic lipid compound, wherein the amount of said fluorinated compound is from 50% to 100% of the amount of fluorinated compound comprised in the initial suspension of step a).

16. The method according to claim 15, wherein said reconstitution does not comprise an additional instillation of fluorinated compound.

17. The method according to claim 15, wherein at step a) said initial suspension is prepared by a method selected from the group consisting of sonication, microbubbles condensation and microfluidic.

18. The method according to claim 15, wherein said initial suspension of nanodroplets comprises a freeze-drying protecting component at a concentration between 1 and 50% (w/v %).

19. (canceled)

20. (canceled)

21. The method according to claim 15, wherein in said initial suspension of nanodroplets the weight ratio between said freeze-drying protecting component and said fluorinated compound is higher than 10, up to 250.

22. The method according to claim 15, wherein the amount of said fluorinated compound into the reconstituted suspension is from 80 to 100% of the amount of fluorinated compound comprised in the initial suspension of step a).

23. A method for preparing a reconstituted suspension of nanodroplets, said method comprising
reconstituting said freeze-dried composition as defined in claim 1 with a pharmaceutically acceptable liquid carrier providing the reconstituted suspension of nanodroplets.

24. The method according to claim 23, wherein the reconstitution of said freeze-dried composition does not comprise an additional instillation of fluorinated compound.

25. A method of diagnostic and/or therapeutic treatment of a patient in need thereof, comprising reconstituting the freeze-dried composition as defined in claim 1 to provide a suspension of nanodroplets and administering said suspension of nanodroplets to the patient.

26. A reconstituted suspension of nanodroplets comprising a freeze-drying protecting component, said nanodroplets comprising an inner core and an outer layer, wherein said inner core comprises a fluorinated compound in liquid form and said outer layer comprises an amphiphilic lipid compound comprising a phospholipid.
